(19) United States
(12) Patent Application Publication
(10) Pub. No.: US 2026/0289743 A1
(43) Pub. Date: Sep. 24, 2026

(54) IMAGE PROCESSING DEVICE AND IMAGE PROCESSING METHOD

(71) Applicant: HAMAMATSU PHOTONICS K.K., Hamamatsu-shi, Shizuoka (JP)

(72) Inventors: Yuya ONISHI, Hamamatsu-shi, Shizuoka (JP); Fumio HASHIMOTO, Hamamatsu-shi, Shizuoka (JP)

(73) Assignee: HAMAMATSU PHOTONICS K.K., Hamamatsu-shi, Shizuoka (JP)

(21) Appl. No.: 18/868,867

(22) PCT Filed: May 25, 2023
(86) PCT No.: PCT/JP2023/019509

§ 371 Date: Nov. 25, 2024

(30) Foreign Application Priority Data
May 31, 2022 (JP) 2022-088585

(51) Int. Cl. G06T 5/70; G06T 5/60; G06T 12/30

(52) U.S. Cl. CPC G06T 5/70; G06T 5/60; G06T 12/30; G06T 2207/10072; G06T 2207/20081

(57) ABSTRACT

An image processing apparatus includes an input image creation unit, a first operation unit, and a second operation unit, and creates a noise reduced image by reducing noise of a target image. The first operation unit includes a first CNN processing unit and a first CNN training unit, and performs processing of supervised pre-learning. A first input image is an image in which pixel values in a partial region are changed based on a training image. The second operation unit includes a second CNN processing unit and a second CNN training unit, and performs processing of unsupervised learning.

### TECHNICAL FIELD

The present disclosure relates to an image processing apparatus and an image processing method for creating a noise reduced image by reducing noise of a target image.

### BACKGROUND ART

An image may contain noise in some cases. An example of the image containing the noise is a tomographic image of a subject which is reconstructed based on information acquired by a radiation tomography apparatus. Examples of the radiation tomography apparatus include a positron emission tomography (PET) apparatus, a single photon emission computed tomography (SPECT) apparatus, and the like.

The PET apparatus includes a detection unit having a large number of small radiation detectors arranged around a measurement space in which the subject is placed. The PET apparatus detects a photon pair of an energy of 511 keV generated by electron positron annihilation in the subject into which a positron-emitting isotope (RI source) is injected by a coincidence method using the detection unit, and collects coincidence information of the detection.

Further, the tomographic image representing a spatial distribution of a generation frequency of the photon pairs in the measurement space (that is, a spatial distribution of the RI sources) can be reconstructed based on the collected many pieces of the coincidence information described above. The above PET apparatus plays an important role in a nuclear medicine field and the like, and can be used to study, for example, a biological function or a brain high-order function.

The reconstructed tomographic image of the subject contains a lot of statistical noise, and thus, it is required to reduce the noise in the tomographic image. Further, in addition to the above PET image (the tomographic image), it may be required to create a noise reduced image by reducing the noise from a target image containing the noise in some cases.

As a noise reduction technique, various techniques are known. In these techniques, a technique for reducing the noise by a deep image prior technique using a convolutional neural network, which is a type of a deep neural network, has attracted attention. Hereinafter, the convolutional neural network is referred to as a “CNN”, and the deep image prior technique is referred to as a “DIP technique”.

In the DIP technique, an image in which the noise is reduced can be created by using unsupervised learning, by taking advantage of the property of the CNN that meaningful structures in the image are learned faster than random noise (that is, the random noise is less likely to be learned).

Non Patent Document 1 describes a technique for reducing the noise in the target image (the PET image) by using the DIP technique. The technique described in this document performs the noise reduction processing in two steps. In the first step, for each of a plurality of sets of a first input image (MRI image) and a training image (PET image), the first input image is input to the CNN to create a first output image by the CNN, and the CNN is trained based on an evaluation result of an error between the first output image and the training image (supervised pre-learning).

In the subsequent second step, a second input image (MRI image) is input to the CNN after the pre-learning in the first step to create a second output image by the CNN, and the CNN is further trained based on an evaluation result of an error between the second output image and the target image (PET image) (unsupervised learning). Further, the second output image is set as the noise reduced image in which the noise of the target image (PET image) is reduced.

As described above, in the noise reduction technique described in Non Patent Document 1, in the first step, the CNN is trained such that the CNN converts the first input image into the training image (the supervised pre-learning), and in the second step, the CNN is further trained (the unsupervised learning), and as a result, the noise reduced image, in which the noise of the target image is reduced, is created. By using the above configuration, the noise reduction technique described in Non Patent Document 1 can improve the noise reduction performance as compared with the case of only the DIP technique (the case of only the unsupervised learning).

### CITATION LIST

### Non Patent Literature

Non Patent Document 1: J. Cui et al., “Populational and individual information based PET image denoising using conditional unsupervised learning”, Phys. Med. Biol. 66 155001, 2021.

### SUMMARY OF INVENTION

### Technical Problem

In the noise reduction technique described in Non Patent Document 1, it is necessary to prepare not only a large number of training images but also a large number of first input images in order to perform the supervised pre-learning in the first step. However, it is not easy to prepare the large number of first input images in addition to the preparation of the large number of training images.

An object of the present invention is to provide an image processing apparatus and an image processing method capable of easily reducing noise of a target image by performing unsupervised learning on a CNN after supervised pre-learning.

### Solution to Problem

An embodiment of the present invention is an image processing apparatus. The image processing apparatus is an image processing apparatus for creating a noise reduced image by reducing noise of a target image, and includes (1) a first CNN processing unit for inputting a first input image, in which pixel values in a partial region are changed based on a training image, to a convolutional neural network, and creating a first output image by the convolutional neural network; (2) a first CNN training unit for evaluating an error between the first output image and the training image, and training the convolutional neural network based on an error evaluation result; (3) a second CNN processing unit for inputting a second input image to the convolutional neural network, and creating a second output image by the convolutional neural network; and (4) a second CNN training unit for evaluating an error between the second output image and the target image, and training the convolutional neural network based on an error evaluation result, and after respective processes of the first CNN processing unit and the first CNN training unit are repeatedly performed a plurality of times for each of a plurality of sets of the training image and the first input image, respective processes of the second CNN processing unit and the second CNN training unit are repeatedly performed a plurality of times, and the second output image is set as the noise reduced image.

An embodiment of the present invention is an image processing method. The image processing method is an image processing method for creating a noise reduced image by reducing noise of a target image, and includes (1) a first CNN processing step of inputting a first input image, in which pixel values in a partial region are changed based on a training image, to a convolutional neural network, and creating a first output image by the convolutional neural network; (2) a first CNN training step of evaluating an error between the first output image and the training image, and training the convolutional neural network based on an error evaluation result; (3) a second CNN processing step of inputting a second input image to the convolutional neural network, and creating a second output image by the convolutional neural network; and (4) a second CNN training step of evaluating an error between the second output image and the target image, and training the convolutional neural network based on an error evaluation result, and after respective processes of the first CNN processing step and the first CNN training step are repeatedly performed a plurality of times for each of a plurality of sets of the training image and the first input image, respective processes of the second CNN processing step and the second CNN training step are repeatedly performed a plurality of times, and the second output image is set as the noise reduced image.

### Advantageous Effects of Invention

According to the embodiments of the present invention, an input image for a convolutional neural network (CNN) used at the time of supervised pre-learning on the CNN can be easily created based on a training image, and further, noise of a target image can be easily reduced by performing unsupervised learning after the supervised pre-learning on the CNN.

### BRIEF DESCRIPTION OF DRAWINGS

FIG. 1 is a diagram illustrating a configuration of an image processing apparatus 1.

FIG. 2 is a flowchart illustrating an image processing method.

FIG. 3 is a diagram illustrating a configuration example of a CNN.

FIG. 4 includes (a)-(c) diagrams each showing an input image (an MRI image) for the CNN.

FIG. 5 includes (a)-(c) diagrams each showing a phantom image (a correct image).

FIG. 6 includes (a)-(c) diagrams each showing a tomographic image (a target image) simulating a head having a tumor and into which 18F-FDG is injected as a drug.

FIG. 7 includes (a)-(c) diagrams each showing a tomographic image after performing noise reduction processing by a image processing method according to a first comparative example.

FIG. 8 includes (a)-(c) diagrams each showing a tomographic image after performing noise reduction processing by a image processing method according to a second comparative example.

FIG. 9 includes (a)-(c) diagrams each showing a tomographic image after performing noise reduction processing by a image processing method according to an example.

FIG. 10 is a table showing respective values of a PSNR, a SSIM, and a CNR of the tomographic images after performing the noise reduction processing by the image processing methods respectively according to the example and the first and second comparative examples.

FIG. 11 includes (a)-(c) diagrams each showing an input image (an MRI image) for the CNN.

FIG. 12 includes (a)-(c) diagrams each showing a tomographic image (a target image) of a head into which 18F-AV45 is injected as a drug.

FIG. 13 includes (a)-(c) diagrams each showing a tomographic image after performing the noise reduction processing by the image processing method according to the first comparative example.

FIG. 14 includes (a)-(c) diagrams each showing a tomographic image after performing the noise reduction processing by the image processing method according to the second comparative example.

FIG. 15 includes (a)-(c) diagrams each showing a tomographic image after performing the noise reduction processing by the image processing method according to the example.

FIG. 16 includes (a), (b) diagrams each showing an input image (an MRI image) for the CNN.

FIG. 17 includes (a), (b) diagrams each showing a tomographic image (a target image) of a head into which 11C-PIB is injected as a drug.

FIG. 18 includes (a), (b) diagrams each showing a tomographic image after performing the noise reduction processing by the image processing method according to the first comparative example.

FIG. 19 includes (a), (b) diagrams each showing a tomographic image after performing the noise reduction processing by the image processing method according to the second comparative example.

FIG. 20 includes (a), (b) diagrams each showing a tomographic image after performing the noise reduction processing by the image processing method according to the example.

FIG. 21 includes (a), (b) diagrams each showing an input image (an MRI image) for the CNN.

FIG. 22 includes (a), (b) diagrams each showing a tomographic image (a target image) of a head into which 18F-FDG is injected as a drug.

FIG. 23 includes (a), (b) diagrams each showing a tomographic image after performing the noise reduction processing by the image processing method according to the first comparative example.

FIG. 24 includes (a), (b) diagrams each showing a tomographic image after performing the noise reduction processing by the image processing method according to the second comparative example.

FIG. 25 includes (a), (b) diagrams each showing a tomographic image after performing the noise reduction processing by the image processing method according the example.

FIG. 26 is a table showing values of the CNR of the tomographic images after performing the noise reduction processing by the image processing methods respectively according to the example and the first and second comparative examples.

### DESCRIPTION OF EMBODIMENTS

Hereinafter, embodiments of an image processing apparatus and an image processing method will be described in detail with reference to the accompanying drawings. In the description of the drawings, the same elements will be denoted by the same reference signs, and redundant description will be omitted. The present invention is not limited to these examples, and the Claims, their equivalents, and all the changes within the scope are intended as would fall within the scope of the present invention.

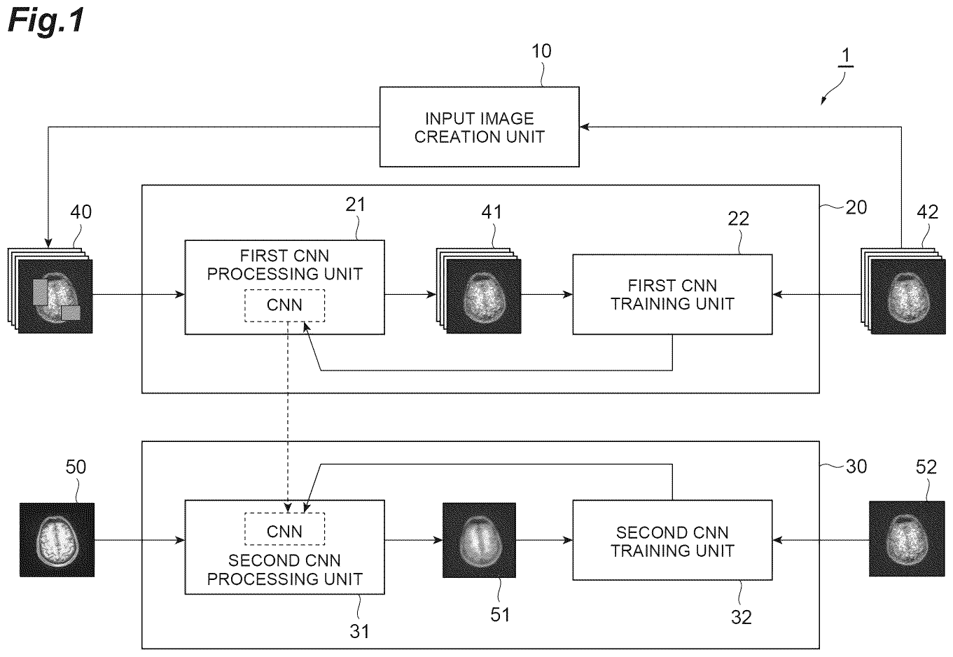

FIG. 1 is a diagram illustrating a configuration of an image processing apparatus 1. The image processing apparatus 1 includes an input image creation unit 10, a first operation unit 20, and a second operation unit 30, and creates a noise reduced image by reducing noise of a target image 52.

In this diagram, a PET image of a head is illustrated as an example of each of a first input image 40, a first output image 41, a training image 42, a second output image 51, and the target image 52, and further, an MRI image of the head is illustrated as an example of a second input image 50. In the following description, it is assumed that the above images are used. In addition, the present embodiment is not limited to the above configuration.

The present embodiment can be suitably used in the case in which a tomographic image (for example, a PET image) of a subject which is reconstructed based on information acquired by a radiation tomography apparatus is set as the target image for reducing noise of the above target image. Each of the above images may be a two-dimensional image, or may be a three-dimensional image.

The image processing apparatus 1 includes a graphics processing unit (GPU) for performing processing by using a CNN, an input unit (for example, a keyboard or a mouse) for receiving an input from an operator, a display unit (for example, a liquid crystal display) for displaying an image and the like, and a storage unit for storing a program and data for executing various types of the processing. As the image processing apparatus 1, a computer including a CPU, a RAM, a ROM, a hard disk drive, and the like is used.

The input image creation unit 10 creates the first input image 40 in which pixel values in a partial region are changed based on the training image 42.

The first operation unit 20 includes a first CNN processing unit 21 and a first CNN training unit 22, and performs processing of the supervised pre-learning. The first CNN processing unit 21 inputs the first input image 40 to the CNN, and creates the first output image 41 by the CNN. The first CNN training unit 22 evaluates an error between the first output image 41 and the training image 42, and trains the CNN based on the error evaluation result.

The first operation unit 20 repeatedly performs the respective processes of the first CNN processing unit 21 and the first CNN training unit 22 a plurality of times for each of a plurality of sets of the training image 42 and the first input image 40.

The second operation unit 30 includes a second CNN processing unit 31 and a second CNN training unit 32, and performs processing of the unsupervised learning. The second CNN processing unit 31 inputs the second input image 50 to the CNN, and creates the second output image 51 by the CNN. The second CNN training unit 32 evaluates an error between the second output image 51 and the target image 52, and trains the CNN based on the error evaluation result.

After the end of the repeated processing performed by the first operation unit 20, the second operation unit 30 repeatedly performs the respective processes of the second CNN processing unit 31 and the second CNN training unit 32 a plurality of times with a training state of the CNN at the time of the above end as an initial value. Further, the second output image 51 at the time of the end of the repeated processing performed by the second operation unit 30 is set as the noise reduced image.

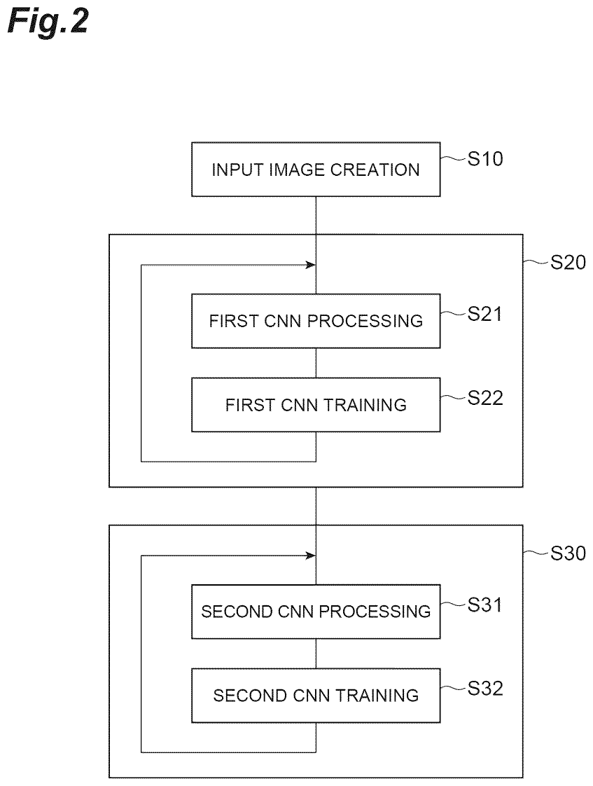

FIG. 2 is a flowchart of an image processing method. The image processing method sequentially performs an input image creation step S10, a first operation step S20, and a second operation step S30 to reduce the noise of the target image 52 and create the noise reduced image.

The input image creation step S10 is a process performed by the input image creation unit 10. In the input image creation step S10, the first input image 40 in which the pixel values in the partial region are changed is created based on the training image 42.

The first operation step S20 is a process of the supervised pre-learning performed by the first operation unit 20, and includes a first CNN processing step S21 and a first CNN training step S22. The first CNN processing step S21 is a process performed by the first CNN processing unit 21. The first CNN training step S22 is a process performed by the first CNN training unit 22.

In the first CNN processing step S21, the first input image 40 is input to the CNN, and the first output image 41 is created by the CNN. In the first CNN training step S22, the error between the first output image 41 and the training image 42 is evaluated, and the CNN is trained based on the error evaluation result. In the first operation step S20, the respective processes of the first CNN processing step S21 and the first CNN training step S22 are repeatedly performed the plurality of times for each of the plurality of sets of the training image 42 and the first input image 40.

The second operation step S30 is a process of the unsupervised learning performed by the second operation unit 30, and includes a second CNN processing step S31 and a second CNN training step S32. The second CNN processing step S31 is a process performed by the second CNN processing unit 31. The second CNN training step S32 is a process performed by the second CNN training unit 32.

In the second CNN processing step S31, the second input image 50 is input to the CNN, and the second output image 51 is created by the CNN. In the second CNN training step S32, the error between the second output image 51 and the target image 52 is evaluated, and the CNN is trained based on the error evaluation result. In the second operation step S30, after the end of the repeated processing performed in the first operation step S20, the respective processes of the second CNN processing step S31 and the second CNN training step S32 are repeatedly performed the plurality of times with the training state of the CNN at the time of the above end as the initial value. Further, the second output image 51 at the time of the end of the repeated processing performed in the second operation step S30 is set as the noise reduced image.

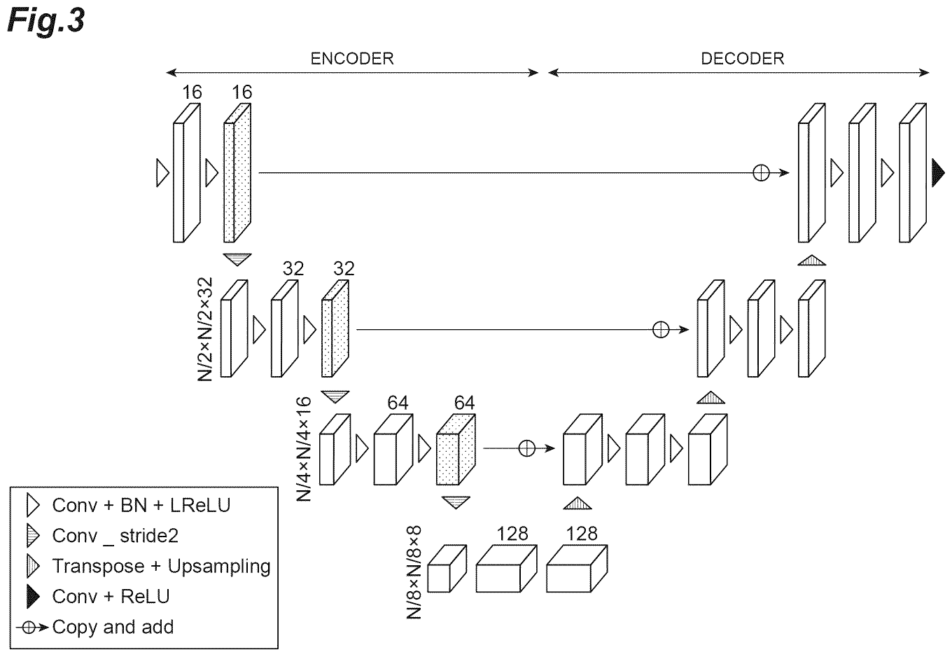

FIG. 3 is a diagram illustrating a configuration example of the CNN. As illustrated in this diagram, it is preferable that the CNN has a U-net structure including an encoder and a decoder. In this diagram, a size of each of the layers of the CNN is illustrated on the assumption that the number of pixels of the input image which is input to the CNN is set to N ×N×64.

In the first CNN training step S22, the first CNN training unit 22 trains both the encoder and the decoder of the CNN. In the second CNN training step S32, the second CNN training unit 32 may train both the encoder and the decoder of the CNN, and in addition, it is preferable to selectively train the decoder out of the encoder and the decoder of the CNN in order to shorten the time required for the training.

In each of the first CNN training unit 22 in the first CNN training step S22 and the second CNN training unit 32 in the second CNN training step S32, a function used for evaluating the error between the two images may be set to an arbitrary function. As the error evaluation function, for example, a L1 norm, a L2 norm, a negative log likelihood in a Poisson distribution, or the like can be used.

In the image processing apparatus 1 or the image processing method, each of the images is as follows. The target image 52 is an image serving as a target in which the noise is to be reduced, and in this case, is set to the PET image of the head. The PET image described above includes, in general, an image region of the head, which is a region of the target of the noise reduction (a noise reduction target region), and a background region around the head region.

The training image 42 is set to the PET image of the head similar to the case of the target image 52. A plurality of training images 42 may be prepared, or one training image 42 may be prepared. The target image 52 itself may be used as the training image 42.

The first input image 40 is an image obtained by changing the pixel values in the partial region based on the training image 42, and is preferably an image obtained by changing the pixel values in the partial region of the noise reduction target region based on the training image 42. A plurality of first input images 40 may be prepared for each training image 42.

The change of the pixel values of the first input image 40 with respect to the training image 42 can be performed by using an arbitrary mode. The partial region in which the pixel values are changed may have an arbitrary shape, an arbitrary size, and an arbitrary number. The change of the pixel values in the partial region may be performed by using, for example, nonlinear conversion of the pixel values, exchange of the pixel values between a plurality of pixels, replacement with certain pixel values, replacement with random pixel values, or the like.

It is preferable that the modes of the change of the pixel values (for example, a shape, a size, and the number of the partial region in which the pixel values are changed, or a method of changing the pixel values) are different from each other in the plurality of first input images 40. In the input image creation step S10, the input image creation unit 10 creates the first input image 40 described above based on the training image 42.

The second input image 50 may be an image representing form information of the subject, may be an MRI image as illustrated in FIG. 1, or may be a CT image or a static PET image. The second input image 50 may be a random noise image.

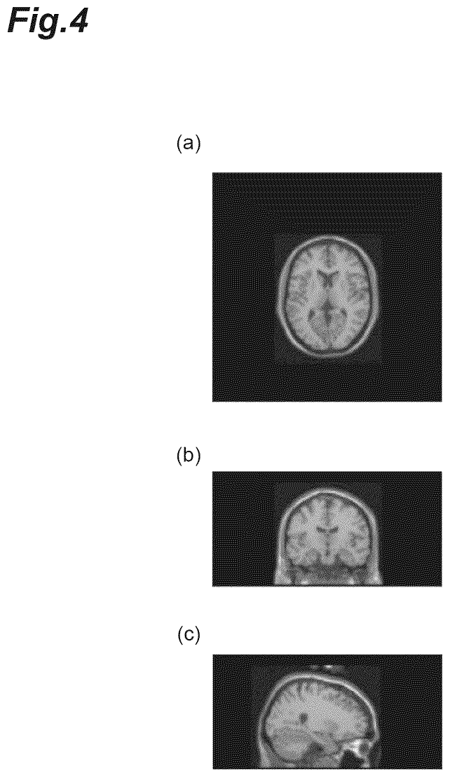

Next, the result obtained by creating the target image by using a Monte Carlo simulation of a head PET apparatus using a digital brain phantom image, and performing the noise reduction processing by the image processing method of each of an example and first and second comparative examples using the target image will be described with reference to FIG. 4 to FIG. 10. The phantom image is obtained from Brain Web (https://brainweb.bic.mni.mcgill.ca/brainweb/).

The image processing method in the example is a method according to the above embodiment. The image processing method in the first comparative example is a method based on the conventional DIP technique. The image processing method in the second comparative example is a method based on the technique described in Non Patent Document 1.

In the supervised pre-learning in the image processing method of each of the example and the second comparative example, 20 training images which are created based on the phantom image are used. In the image processing method of the example, 32 first input images which are created by variously changing the pixel value change mode for each training image are used. In the image processing method of the second comparative example, an MRI image is used as the input image which is input to the CNN.

In the unsupervised learning in the image processing method of each of the example and the first and second comparative examples, a tomographic image simulating a head having a tumor and into which 18F-FDG is injected as a drug is used as the target image. An MRI image is used as the input image which is input to the CNN.

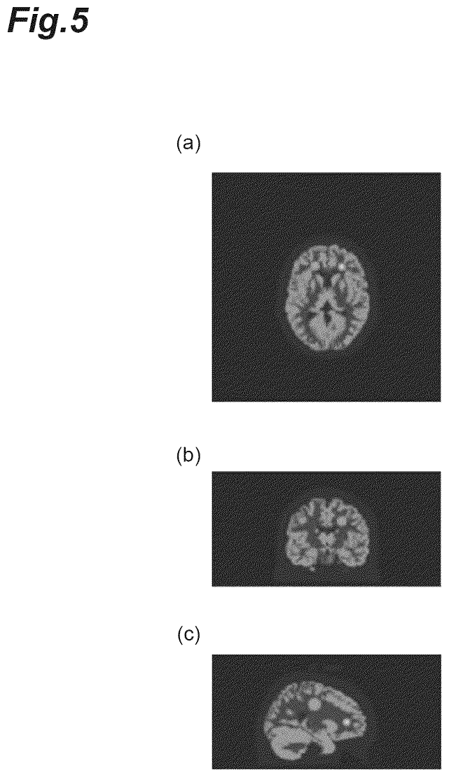

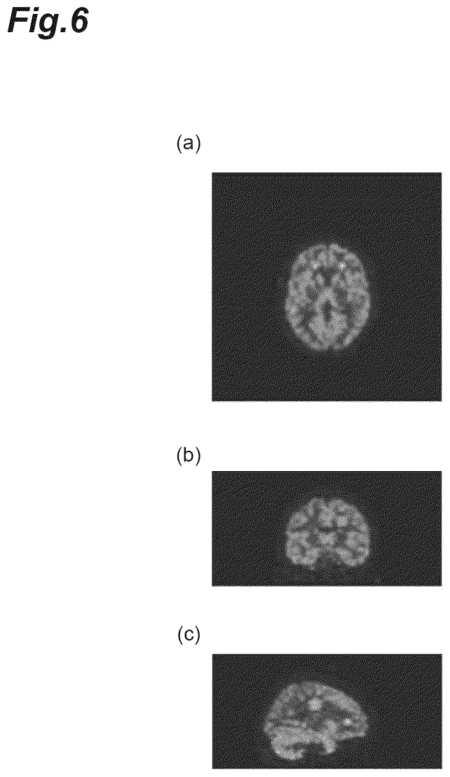

Each of FIG. 4 to FIG. 9 includes diagrams each showing the tomographic image used or created in the unsupervised learning in the image processing method of each of the example and the first and second comparative examples. FIG. 4 includes diagrams each showing the input image (the MRI image) for the CNN. FIG. 5 includes diagrams each showing the phantom image (the correct image). FIG. 6 includes diagrams each showing the tomographic image (the target image) simulating the head having the tumor and into which 18F-FDG is injected as the drug.

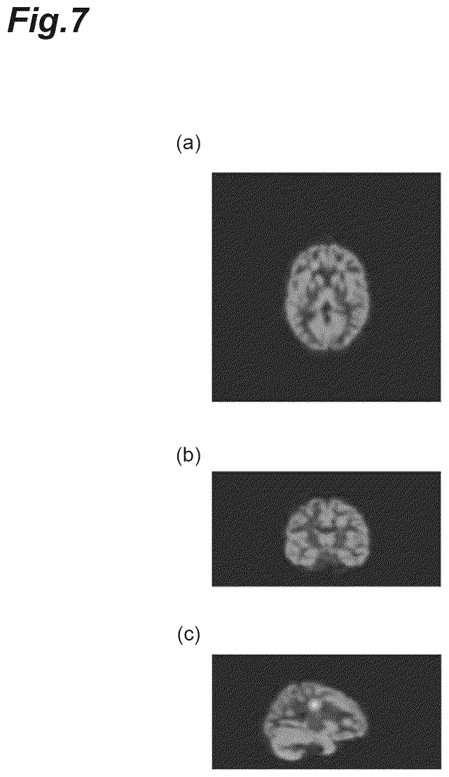

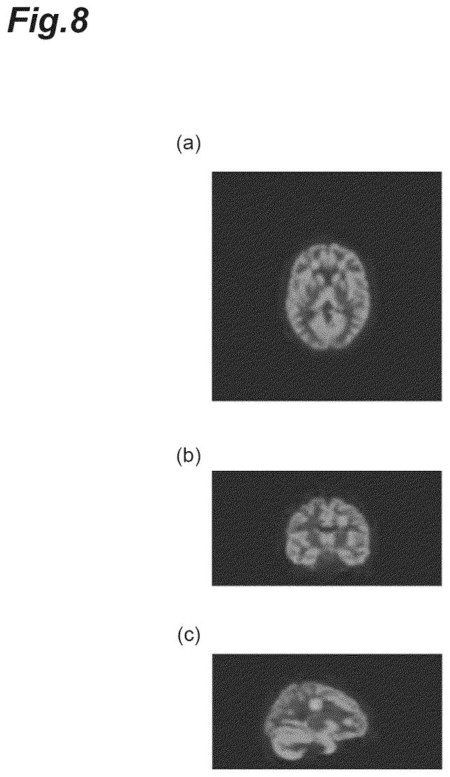

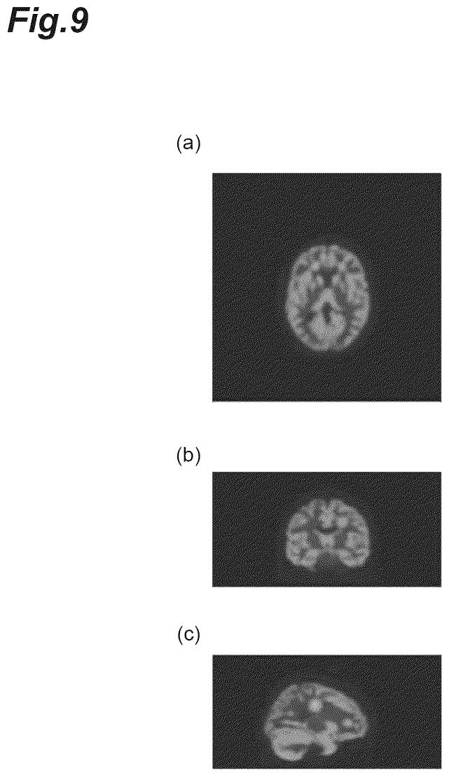

FIG. 7 includes diagrams each showing the tomographic image after performing the noise reduction processing by the image processing method according to the first comparative example. FIG. 8 includes diagrams each showing the tomographic image after performing the noise reduction processing by the image processing method according to the second comparative example. FIG. 9 includes diagrams each showing the tomographic image after performing the noise reduction processing by the image processing method according to the example. In each of FIG. 4 to FIG. 9, (a) shows an image of a transverse section, (b) shows an image of a coronal section, and (c) shows an image of a sagittal section.

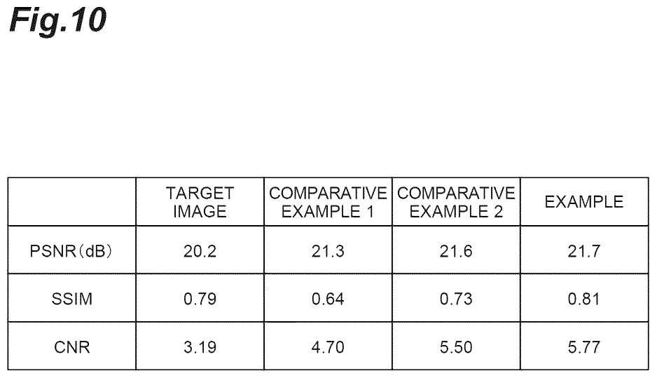

FIG. 10 is a table showing respective values of a PSNR, a SSIM, and a CNR of the tomographic images after performing the noise reduction processing by the image processing methods respectively according to the example and the first and second comparative examples. The peak signal to noise ratio (PSNR) represents the quality of the image in decibel (dB). The structural similarity index (SSIM) is an index for quantifying the change of the brightness, the contrast, and the structure of the image. The contrast to noise ratio (CNR) is a ratio of the contrast and the noise of the image. In each of the PSNR, the SSIM, and the CNR, the larger the value is, the better the image quality is.

As shown in FIG. 4 to FIG. 10, in the example, the values of the PSNR, the SSIM, and the CNR are respectively larger than the values in the first and second comparative examples, and it can be confirmed that the noise of the target image can be reduced more effectively.

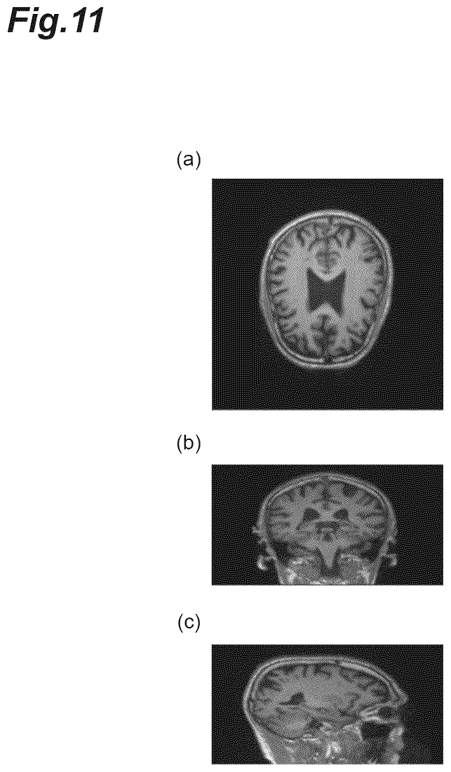

Next, the result obtained by performing the noise reduction processing by the image processing method of each of the example and the first and second comparative examples using clinical data will be described with reference to FIG. 11 to FIG. 26.

In the supervised pre-learning in the image processing method of each of the example and the second comparative example, 24 tomographic images of the clinical data are used as the training image. Each of the above tomographic images is a PET image of a head into which 18F-AV45 is injected as a drug. In the image processing method of the example, 32 first input images which are created by variously changing the pixel value change mode for each training image are used. In the image processing method of the second comparative example, an MRI image is used as the input image which is input to the CNN.

In the unsupervised learning in the image processing method of each of the example and the first and second comparative examples, a tomographic image of a head into which any of 18F-AV45, 11C-PIB, and 18F-FDG is injected as a drug is used as the target image. An MRI image is used as the input image which is input to the CNN.

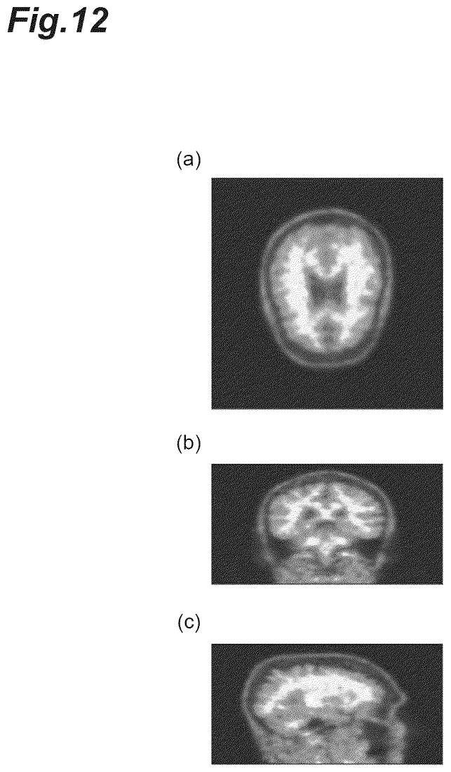

Each of FIG. 11 to FIG. 15 includes diagrams each showing the tomographic image used or created in the unsupervised learning in the image processing method of each of the example and the first and second comparative examples, in the case in which the tomographic image of the head into which 18F-AV45 is injected as the drug is used as the target image. FIG. 11 includes diagrams each showing the input image (the MRI image) for the CNN. FIG. 12 includes diagrams each showing the tomographic image (the target image) of the head into which 18F-AV45 is injected as the drug.

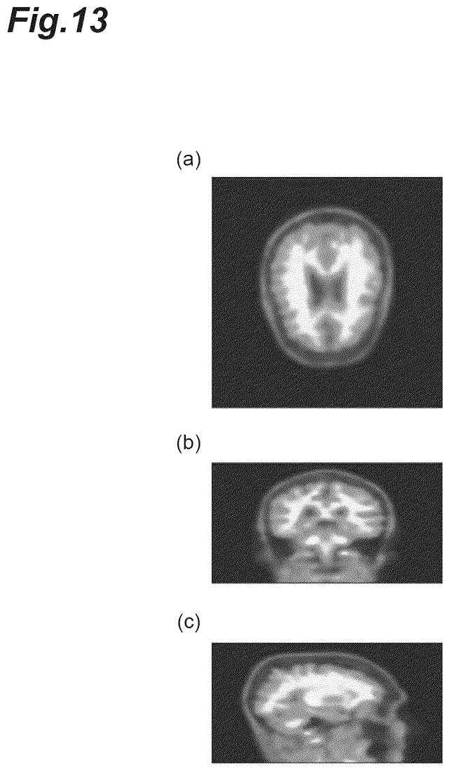

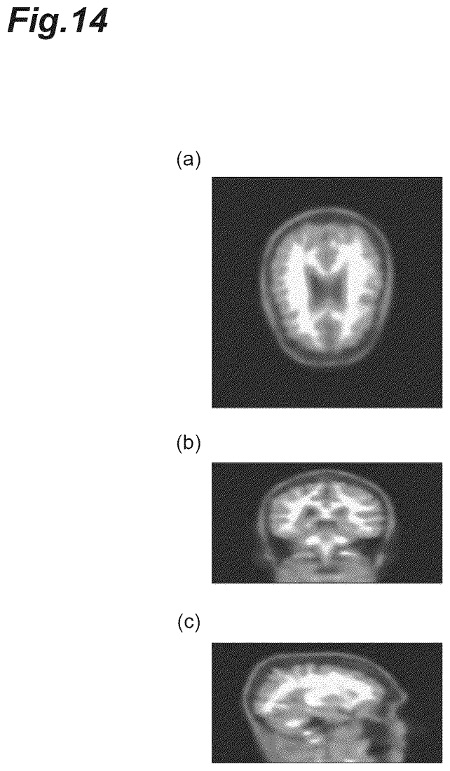

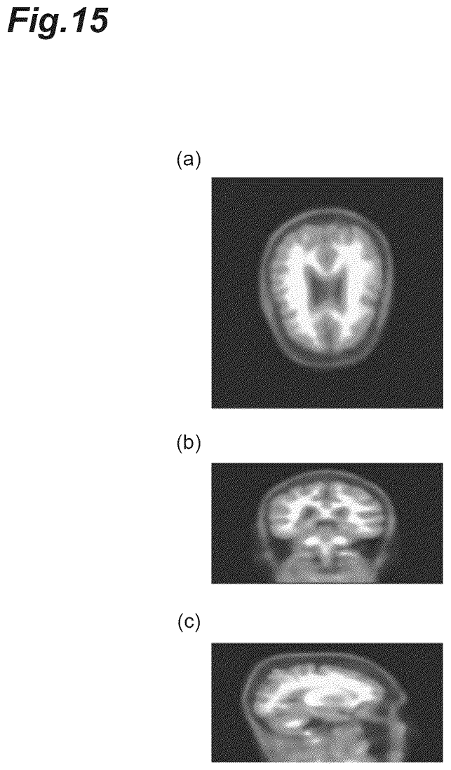

FIG. 13 includes diagrams each showing the tomographic image after performing the noise reduction processing by the image processing method according to the first comparative example. FIG. 14 includes diagrams each showing the tomographic image after performing the noise reduction processing by the image processing method according to the second comparative example. FIG. 15 includes diagrams each showing the tomographic image after performing the noise reduction processing by the image processing method according to the example. In each of FIG. 11 to FIG. 15, (a) shows an image of a transverse section, (b) shows an image of a coronal section, and (c) shows an image of a sagittal section.

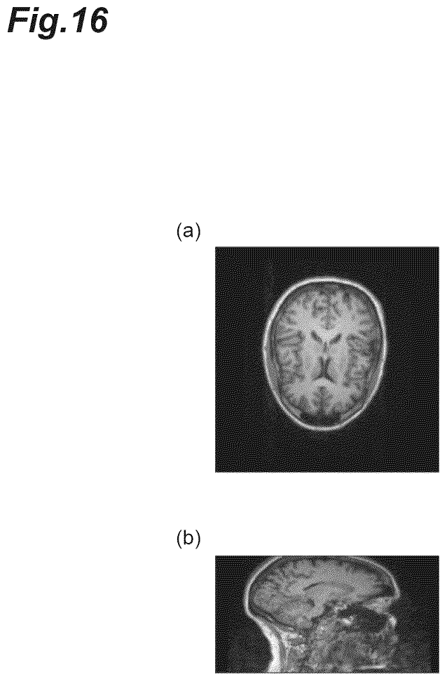

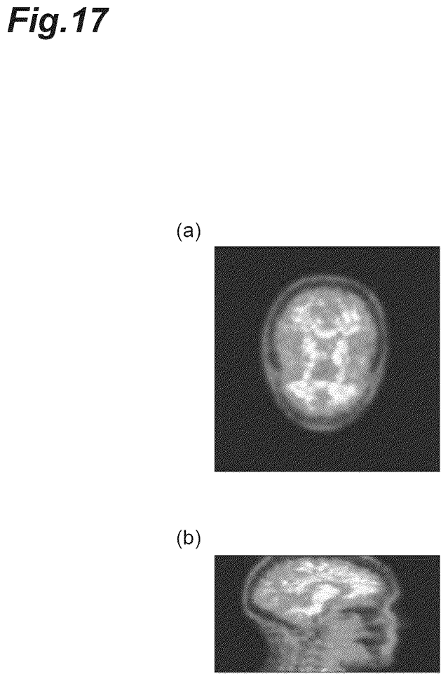

Each of FIG. 16 to FIG. 20 includes diagrams each showing the tomographic image used or created in the unsupervised learning in the image processing method of each of the example and the first and second comparative examples, in the case in which the tomographic image of the head into which 11C-PIB is injected as the drug is used as the target image. FIG. 16 includes diagrams each showing the input image (the MRI image) for the CNN. FIG. 17 includes diagrams each showing the tomographic image (the target image) of the head into which 11C-PIB is injected as the drug.

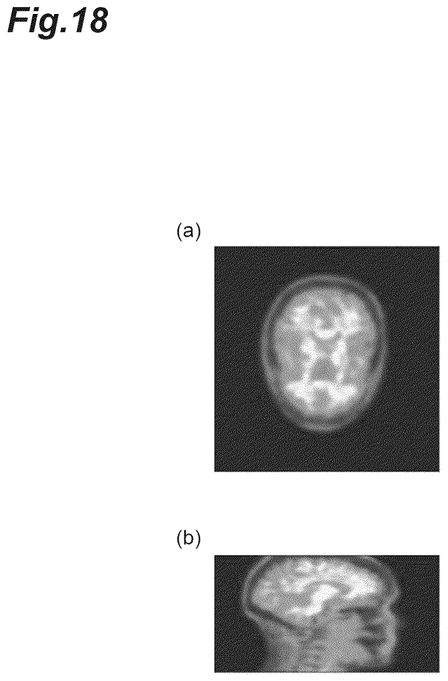

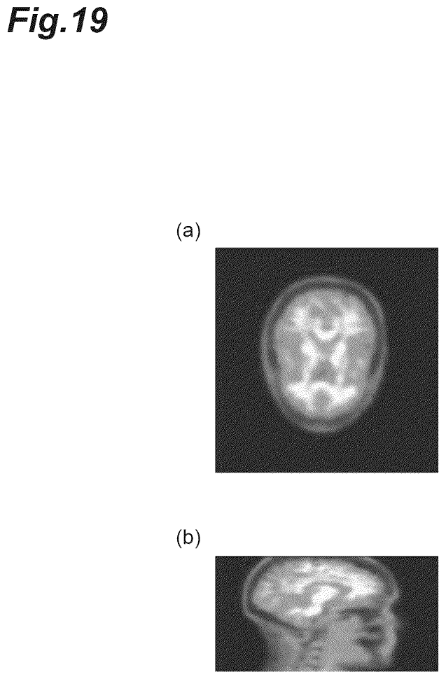

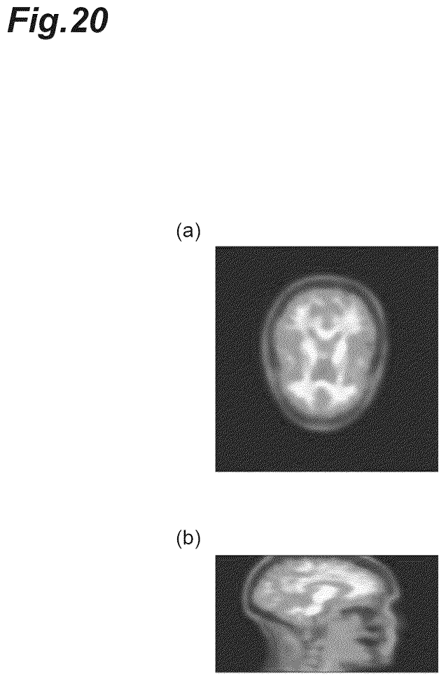

FIG. 18 includes diagrams each showing the tomographic image after performing the noise reduction processing by the image processing method according to the first comparative example. FIG. 19 includes diagrams each showing the tomographic image after performing the noise reduction processing by the image processing method according to the second comparative example. FIG. 20 includes diagrams each showing the tomographic image after performing the noise reduction processing by the image processing method according to the example. In each of FIG. 16 to FIG. 20, (a) shows an image of a transverse section, and (b) shows an image of a sagittal section.

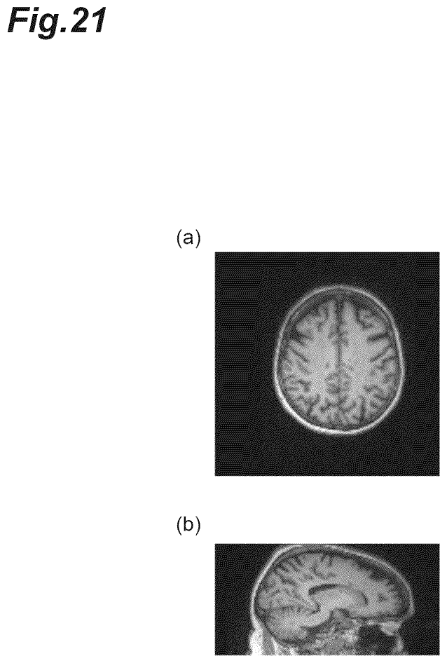

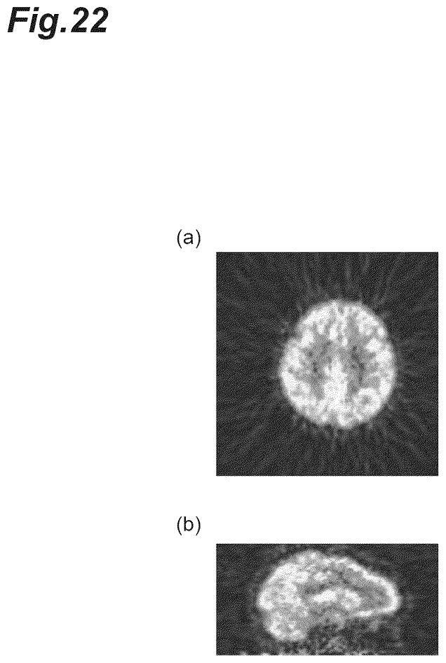

Each of FIG. 21 to FIG. 25 includes diagrams each showing the tomographic image used or created in the unsupervised learning in the image processing method of each of the example and the first and second comparative examples, in the case in which the tomographic image of the head into which 18F-FDG is injected as the drug is used as the target image. FIG. 21 includes diagrams each showing the input image (the MRI image) for the CNN. FIG. 22 includes diagrams each showing the tomographic image (the target image) of the head into which 18F-FDG is injected as the drug.

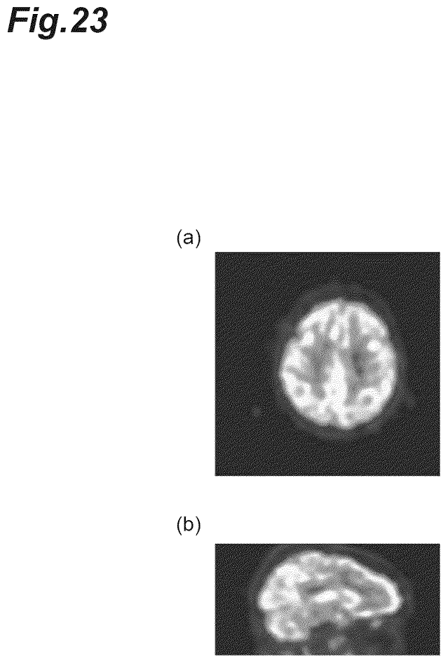

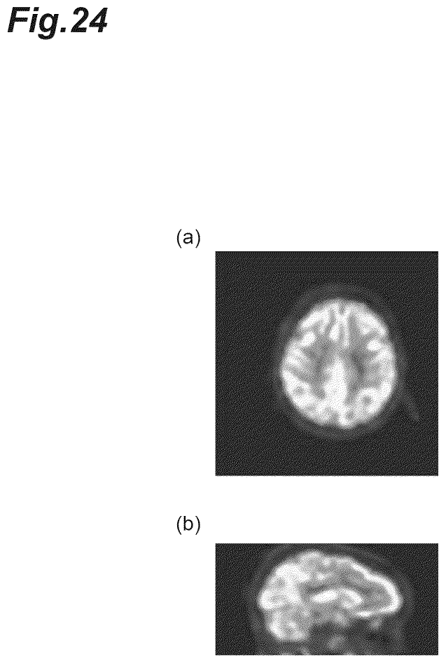

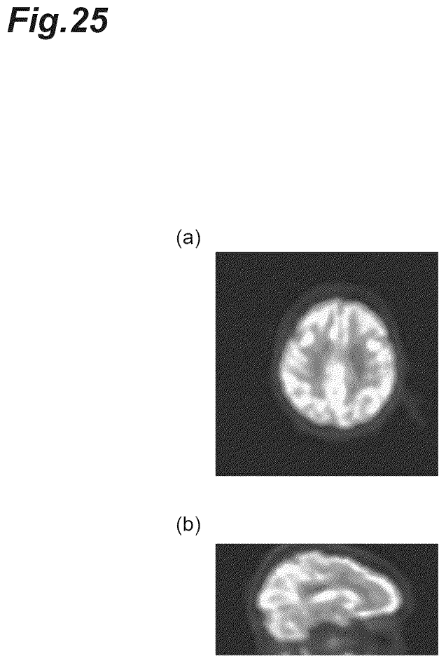

FIG. 23 includes diagrams each showing the tomographic image after performing the noise reduction processing by the image processing method according to the first comparative example. FIG. 24 includes diagrams each showing the tomographic image after performing the noise reduction processing by the image processing method according to the second comparative example. FIG. 25 includes diagrams each showing the tomographic image after performing the noise reduction processing by the image processing method according to the example. In each of FIG. 21 to FIG. 25, (a) shows an image of a transverse section, and (b) shows an image of a sagittal section.

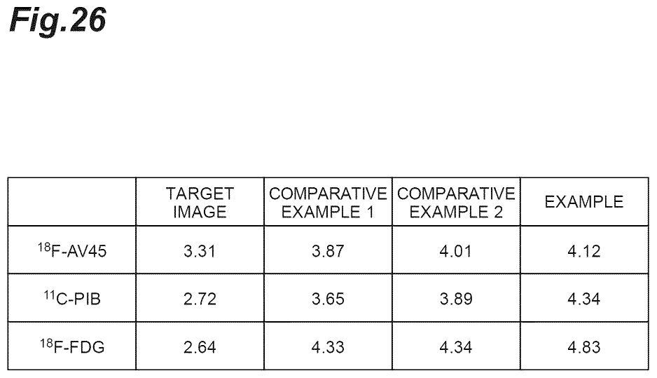

FIG. 26 is a table showing values of the CNR of the tomographic images after performing the noise reduction processing by the image processing methods respectively according to the example and the first and second comparative examples. In this table, the values of the CNR are shown for the cases in which 18F-AV45, 11C-PIB, and 18F-FDG are respectively injected into the head as the drug.

The following can be seen from FIG. 11 to FIG. 26. In the example, the values of the CNR are larger than the values in the first and second comparative examples, and it can be confirmed that the noise of the target image can be reduced more effectively. In the case in which the tomographic image when 18F-FDG is injected into the head as the drug is used as the target image, the degree of improvement of the CNR is small in the second comparative example as compared with the first comparative example, and on the other hand, the degree of improvement of the CNR is large in the example as compared with the first and second comparative examples.

In this way, in the case in which the type of the drug injected into the head is different between the training image and the target image, the degree of noise removal in the second comparative example is the same level as that in the first comparative example, and on the other hand, it is confirmed that the degree of noise removal in the example is improved as compared with that in the first and second comparative examples.

As described above, according to the present embodiment, the input image for the CNN used at the time of the supervised pre-learning on the CNN can be easily created based on the training image, and thus, it is possible to easily reduce the noise of the target image by performing the unsupervised learning after the supervised pre-learning on the CNN. Further, even in the case in which the type of the drug injected into the subject is different between the training image and the target image, it is possible to effectively reduce the noise of the target image.

The image processing apparatus and the image processing method are not limited to the embodiments and configuration examples described above, and various modifications are possible.

The image processing apparatus of a first aspect according to the above embodiment is an image processing apparatus for creating a noise reduced image by reducing noise of a target image, and includes (1) a first CNN processing unit for inputting a first input image, in which pixel values in a partial region are changed based on a training image, to a convolutional neural network, and creating a first output image by the convolutional neural network; (2) a first CNN training unit for evaluating an error between the first output image and the training image, and training the convolutional neural network based on an error evaluation result; (3) a second CNN processing unit for inputting a second input image to the convolutional neural network, and creating a second output image by the convolutional neural network; and (4) a second CNN training unit for evaluating an error between the second output image and the target image, and training the convolutional neural network based on an error evaluation result, and after respective processes of the first CNN processing unit and the first CNN training unit are repeatedly performed a plurality of times for each of a plurality of sets of the training image and the first input image, respective processes of the second CNN processing unit and the second CNN training unit are repeatedly performed a plurality of times, and the second output image is set as the noise reduced image.

In the image processing apparatus of a second aspect, in the configuration of the first aspect, the apparatus may further include an input image creation unit for creating the first input image in which the pixel values in the partial region are changed based on the training image.

In the image processing apparatus of a third aspect, in the configuration of the first or second aspect, the convolutional neural network may have a U-net structure including an encoder and a decoder, the first CNN training unit may train both the encoder and the decoder of the convolutional neural network, and the second CNN training unit may selectively train the decoder of the convolutional neural network.

In the image processing apparatus of a fourth aspect, in the configuration of any one of the first to third aspects, the first CNN processing unit may input, for each training image, a plurality of first input images with modes of changing the pixel values different from each other respectively to the convolutional neural network, and may create the first output image by the convolutional neural network for each first input image.

In the image processing apparatus of a fifth aspect, in the configuration of any one of the first to fourth aspects, the first CNN processing unit may input the first input image, in which the pixel values in the partial region of a noise reduction target region are changed based on the training image, to the convolutional neural network.

In the image processing apparatus of a sixth aspect, in the configuration of any one of the first to fifth aspects, each of the target image and the training image may be a tomographic image of a subject reconstructed based on information acquired by a radiation tomography apparatus.

The image processing method of a first aspect according to the above embodiment is an image processing method for creating a noise reduced image by reducing noise of a target image, and includes (1) a first CNN processing step of inputting a first input image, in which pixel values in a partial region are changed based on a training image, to a convolutional neural network, and creating a first output image by the convolutional neural network; (2) a first CNN training step of evaluating an error between the first output image and the training image, and training the convolutional neural network based on an error evaluation result; (3) a second CNN processing step of inputting a second input image to the convolutional neural network, and creating a second output image by the convolutional neural network; and (4) a second CNN training step of evaluating an error between the second output image and the target image, and training the convolutional neural network based on an error evaluation result, and after respective processes of the first CNN processing step and the first CNN training step are repeatedly performed a plurality of times for each of a plurality of sets of the training image and the first input image, respective processes of the second CNN processing step and the second CNN training step are repeatedly performed a plurality of times, and the second output image is set as the noise reduced image.

In the image processing method of a second aspect, in the configuration of the first aspect, the method may further include an input image creation step of creating the first input image in which the pixel values in the partial region are changed based on the training image.

In the image processing method of a third aspect, in the configuration of the first or second aspect, the convolutional neural network may have a U-net structure including an encoder and a decoder, in the first CNN training step, both the encoder and the decoder of the convolutional neural network may be trained, and in the second CNN training step, the decoder of the convolutional neural network may be selectively trained.

In the image processing method of a fourth aspect, in the configuration of any one of the first to third aspects, in the first CNN processing step, for each training image, a plurality of first input images with modes of changing the pixel values different from each other may be respectively input to the convolutional neural network, and the first output image may be created by the convolutional neural network for each first input image.

In the image processing method of a fifth aspect, in the configuration of any one of the first to fourth aspects, in the first CNN processing step, the first input image, in which the pixel values in the partial region of a noise reduction target region are changed based on the training image, may be input to the convolutional neural network.

In the image processing method of a sixth aspect, in the configuration of any one of the first to fifth aspects, each of the target image and the training image may be a tomographic image of a subject reconstructed based on information acquired by a radiation tomography apparatus.

### Industrial Applicability

The present invention can be used as an image processing apparatus and an image processing method capable of easily reducing noise of a target image by performing unsupervised learning after supervised pre-learning on a CNN.

### Reference Signs List

1—image processing apparatus, 10—input image creation unit, 20—first operation unit, 21—first CNN processing unit, 22—first CNN training unit, 30—second operation unit, 31—second CNN processing unit, 32—second CNN training unit.

1. An image processing apparatus for creating a noise reduced image by reducing noise of a target image, the image processing apparatus comprising:
a first CNN processing unit configured to input a first input image, in which pixel values in a partial region are changed based on a training image, to a convolutional neural network, and create a first output image by the convolutional neural network;
a first CNN training unit configured to evaluate an error between the first output image and the train image, and training the convolutional neural network based on an error evaluation result;
a second CNN processing unit configured to input a second input image to the convolutional neural network, and create a second output image by the convolutional neural network; and
a second CNN training unit configured to evaluate an error between the second output image and the target image, and train the convolutional neural network based on an error evaluation result, wherein
after respective processes of the first CNN processing unit and the first CNN training unit are repeatedly performed a plurality of times for each of a plurality of sets of the training image and the first input image, respective processes of the second CNN processing unit and the second CNN training unit are repeatedly performed a plurality of times, and the second output image is set as the noise reduced image.

2. The image processing apparatus according to claim 1, further comprising an input image creation unit configured to create the first input image in which the pixel values in the partial region are changed based on the training image.

3. The image processing apparatus according to claim 1, wherein the convolutional neural network has a U-net structure including an encoder and a decoder,
the first CNN training unit is configured to train both the encoder and the decoder of the convolutional neural network, and
the second CNN training unit is configured to selectively train the decoder of the convolutional neural network.

4. The image processing apparatus according to claim 1, wherein the first CNN processing unit is configured to input, for each training image, a plurality of first input images with modes of changing the pixel values different from each other respectively to the convolutional neural network, and create the first output image by the convolutional neural network for each first input image.

5. The image processing apparatus according to claim 1, wherein the first CNN processing unit is configured to input the first input image, in which the pixel values in the partial region of a noise reduction target region are changed based on the training image, to the convolutional neural network.

6. The image processing apparatus according to claim 1, wherein each of the target image and the training image is a tomographic image of a subject reconstructed based on information acquired by a radiation tomography apparatus.

7. An image processing method for creating a noise reduced image by reducing noise of a target image, the image processing method comprising:
a first CNN processing step of inputting a first input image, in which pixel values in a partial region are changed based on a training image, to a convolutional neural network, and creating a first output image by the convolutional neural network;
a first CNN training step of evaluating an error between the first output image and the training image, and training the convolutional neural network based on an error evaluation result;
a second CNN processing step of inputting a second input image to the convolutional neural network, and creating a second output image by the convolutional neural network; and
a second CNN training step of evaluating an error between the second output image and the target image, and training the convolutional neural network based on an error evaluation result, wherein
after respective processes of the first CNN processing step and the first CNN training step are repeatedly performed a plurality of times for each of a plurality of sets of the training image and the first input image, respective processes of the second CNN processing step and the second CNN training step are repeatedly performed a plurality of times, and the second output image is set as the noise reduced image.

8. The image processing method according to claim 7, further comprising an input image creation step of creating the first input image in which the pixel values in the partial region are changed based on the training image.

9. The image processing method according to claim 7, wherein the convolutional neural network has a U-net structure including an encoder and a decoder,
in the first CNN training step, both the encoder and the decoder of the convolutional neural network are trained, and
in the second CNN training step, the decoder of the convolutional neural network is selectively trained.

10. The image processing method according to claim 7, wherein in the first CNN processing step, for each training image, a plurality of first input images with modes of changing the pixel values different from each other are respectively input to the convolutional neural network, and the first output image is created by the convolutional neural network for each first input image.

11. The image processing method according to claim 7, wherein in the first CNN processing step, the first input image, in which the pixel values in the partial region of a noise reduction target region are changed based on the training image, is input to the convolutional neural network.

12. The image processing method according to claim 7, wherein each of the target image and the training image is a tomographic image of a subject reconstructed based on information acquired by a radiation tomography apparatus.
